(19) United States
(12) Plant Patent Application Publication
(10) Pub. No.: US 2017/0112038 P1
(43) Pub. Date: Apr. 20, 2017

(54) Lachenalia jacq.f. ex murray named "RIANA"

(50) ;

(71) Applicant: AGRICULTURAL RESEARCH COUNCIL, Pretoria (ZA)

(72) Inventor: Riana KLEYNHANS, Benoni (ZA)

(21) Appl. No.: 14/756,819

(22) Filed: Oct. 19, 2015

(51) Int. Cl. A01H 5/00

(52) U.S. Cl. PLT/263.1

(57) ABSTRACT

A new and distinct form of Lachenalia plant characterized by a bulb plant type having a spreading growth habit of a height of 15-30 centimetres (cm) and width of 10-15 cm having a hyacinth-like soft pink flower with spreading tips; green leave with maroon spots, further characterized in that the spots are fairly pronounced in that it has a good contrast.

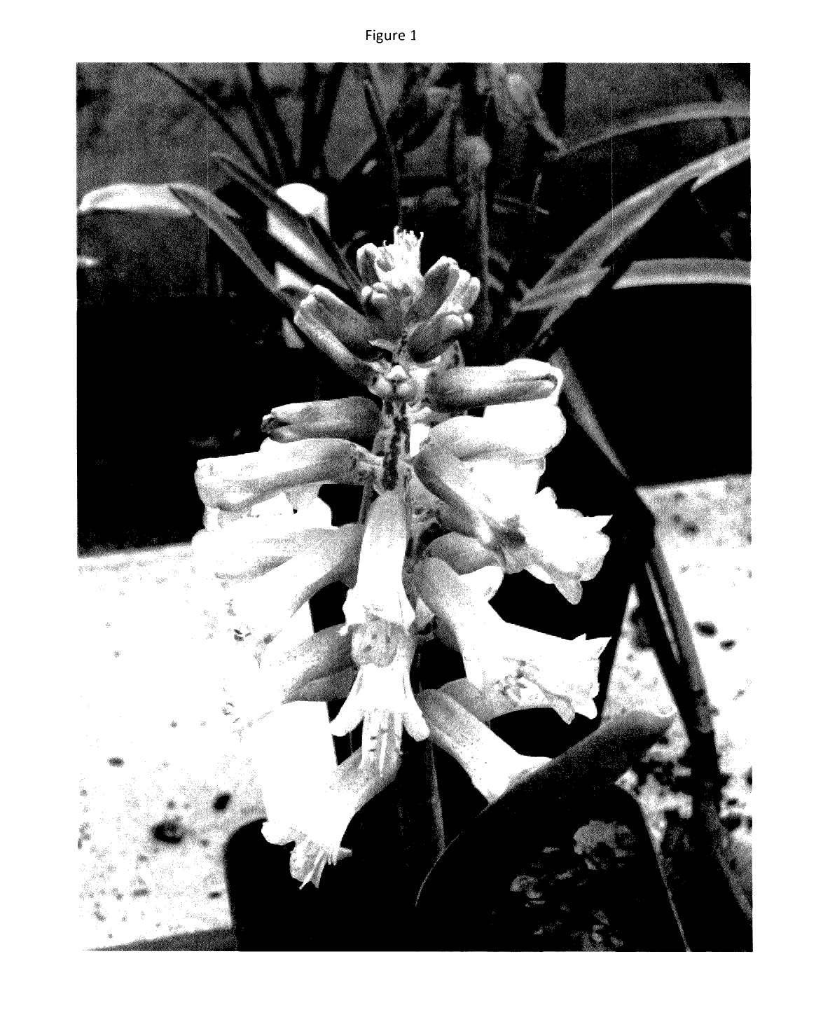

Genus and species: Lachenalia Jacq. f. ex Murray.

Variety denomination: Riana.

### BACKGROUND OF THE INVENTION

The present invention relates to a new and distinct cultivar of Lachenalia, botanically known as Lachenalia Jacq. f. ex Murray having the common name of Viooltjies/Cape cowslip and hereinafter referred to by its variety denomination “Riana”.

The variety was first cultivated in 2008 and originated by means of conventional breeding by the inventor during 2009 to 2010 at the premises of Agricultural Research Council Vegetable and Ornamental Plant Institute, Roodeplaat, Pretoria, Republic of South Africa.

Asexual reproduction of the Riana plant has shown that the unique features thereof are stable and reproduced true to type in successive generations.

### SUMMARY OF THE INVENTION

The present invention constitutes a new and distinct variety of Lachenalia which originated from the controlled pollination between a Lachenalia hybrid as seed bearing parent and a Lachenalia viridiflora as pollen parent as detailed hereinbelow.

The following traits have been repeatedly observed and are determined to be the unique characteristics, under the local evaluation conditions, of the Riana plant. These characteristics in combination distinguish Riana as a new and distinct Lachenalia cultivar:
1. Bulb plant type having a spreading growth habit of a height of 15-30 centimetres (cm) and width of 10-15 cm.
2. Flower color: soft pink.
3. Leaf color: green with maroon spots.
4. Flower form: Hyacinth like with spreading tips.

Plants of the new Lachenalia variety Riana can be compared to similar denominations such as Rupert and Rolina but differs in that the flower color is a soft pink, compared to the purple and apricot colors of Rupert and Rolina respectively. The flower form of Riana is hyacinth-like with spreading tips compared to the tubular form of Rolina. The maroon spots on the leafs of the Riana plant is furthermore more pronounced in that it has a good contrast compared to the low contrast found in Rupert.

### BRIEF DESCRIPTION OF THE PHOTOGRAPH

The accompanying colored photograph illustrates the overall appearance of the Riana, showing the colors as true as it is reasonably possible to obtain in colored reproductions of this type. The photograph comprises a front perspective view of a typical Riana grown in an indoor nursery.

### DETAILED DESCRIPTION

The aforementioned photograph, following observations and measurements describe Riana grown by the inventor on behalf of Agricultural Research Council Vegetable and Ornamental Plant Institute, Roodeplaat, Pretoria, Republic of South Africa. Thereafter Riana was made subject of a South African Plant Breeders' Right application in March 2009. This application was granted in 2010 under number ZA20104397.

The plant has an intermediate flower time and may be used for landscaping and should be planted during the month of March; indoors and in pots only. The plant should be kept at temperatures of 20-25 degrees Celsius during the day and 10-15 degrees Celsius at night. Plenty of light should be provided.

Riana is a hybrid-hybrid cross containing the following Lachenalia species: Lachenalia orchioides subsp. glaucina, Lachenalia bulbifera, Lachenalia quadricolor, Lachenalia aloides and Lachenalia rubida.
Leaf: Monocolored.
   Spots on upper side.—Present, small and with good contrast.
   Attitude.—Semi-erect.
   Length.—Medium to long — 25 cm.
   Width.—Medium to wide — 5.7 cm.
   Ratio.—length/width. Large (elongated).
   Shape.—Oblong.
   Color.—Dark green.
   Shape in cross section.—Angular.
   Recurving and undulation of margin.—Absent.
   Blistering and pubescence of upper side.—Absent.
   Spots on upper side.—Present (Purplish).
   Size of spots on upper side.—Large.
   Density of spots on upper side.—Medium.
   Intensity of spots on upper side.—Light to medium.
   Markings on outer side of leaf base.—Present.
Peduncle:
   Length.—Medium — 17 cm.
   Spots or markings.—Present.
   Size of spots or markings.—Large.
   Density of spots or markings.—Dense.
Interflorescense:
   Length.—Medium to long — 22 cm.
   Rudimentary apex.—Conspicuous.
   Number of flowers.—Many.
Flower:
   Attitude.—Horizontal.
   Pedicel.—Present.
   Length of pedicel.—Short to medium.
   Length of flower.—Medium — 2.7 cm.
   Diameter excluding apex.—Medium — 0.8 cm.
   Diameter at apex.—Large — 1.9 cm.
   Attitude at distal part of inner perianth segments.—Spreading.
   Glossiness.—Absent.
   Predominant color.—Pink.
   Intensity of predominant color.—Medium to strong.
   Outer perianth segment.—Color of basal part just before opening of flower. Red-purple (plum) (RHS #70B).
   Outer perianth segment.—Color of basal part of fully opened flower. Red-purple (RHS #70C).
   Outer perianth segment.—Color of apex relative to other part. Clearly different.
   Outer perianth segment.—Color of apex if clearly different from other part . Yellow (cream).
   Inner perianth segment.—Distinct spot in middle of apex. Present.
   Inner perianth segment.—Color of margin of apex relative to other part. Similar.
   Inner perianth segment.—Color of differently colored margin of apex. Other (light pink).
   Inner perianth segment.—Predominant color of exposed part (excluding differently colored margin of apex). Yellow (cream) (RHS #4C).
   Length of inner perianth segment compared to outer segments.—Longer.
   Extrusion of stamens.—Absent.
   Fragrance.—Absent.
   Predominant shape.—Oblate.
   Time of flowering.—Medium.
   Duration of flowering.—Long. The variety is not genetically modified and has been tested to be free of Ornithogalum Mosaic Virus.

The invention claimed is:

1. A new and distinct cultivar of Lachenalia named Riana as illustrated and described herein.
